(19) United States
(12) Patent Application Publication
(10) Pub. No.: US 2006/0280815 A1
(43) Pub. Date: Dec. 14, 2006

(54) Nutritional composition which promotes weight loss, burns calories, increases thermogenesis, supports energy metabolism and/or suppresses appetite

(76) Inventors: Paul T. Gardiner, Mississauga (CA); Marvin A. Heuer, Etobicoke (CA)

Correspondence Address: KENYON & KENYON LLP, ONE BROADWAY, NEW YORK, NY, 10004, US

(21) Appl. No.: 11/506,711

(22) Filed: Aug. 18, 2006

Related U.S. Application Data
(62) Division of application No. 11/117,942, filed on Apr. 29, 2005.
(60) Provisional application No. 60/567,126, filed on Apr. 30, 2004.

(51) Int. Cl. A61K 36/82; A61K 36/54; A61K 36/48; A61K 36/58; A61K 31/555

(52) U.S. Cl. 424/729; 424/761; 424/778; 514/184; 424/739; 424/757; 424/774

(57) ABSTRACT

The present invention provides for a nutritional composition including at least garcinia cambogia extract, gymnema sylvestre leaf extract, and green tea leaf extract, consumption of the nutritional composition providing a method of inducing fast weight loss, burning calories, increasing thermogenesis, supports energy metabolism and/or suppressing appetite in individuals. The nutritional composition may be suitable for individuals on a low carbohydrate diet, for individuals on a reduced caffeine or caffeine-free dietary regimen and/or for individuals on a diet of natural ingredients. The present invention may also provide for a method of manufacturing the nutritional composition.

### RELATED APPLICATIONS

This application is related to and claims the benefit of priority to U.S. Provisional Patent Application Ser. No. 60/567,126, entitled “Nutritional Composition for Weight Loss,” filed on Apr. 30, 2004, the disclosure of which is hereby fully incorporated by reference herein.

### FIELD OF THE INVENTION

The present invention relates to a novel nutritional composition, which promotes fast weight loss, burns calories, increases thermogenesis, supports energy metabolism and/or suppresses appetite in individuals.

### SUMMARY OF THE INVENTION

The present invention provides for a novel nutritional composition which promotes fast weight loss, burns calories, increases thermogenesis, supports energy metabolism and/or suppresses appetites in individuals. In one embodiment, the nutritional composition includes garcinia cambogia extract, gymnema sylvestre leaf extract, and green tea leaf extract. In one embodiment, the present invention provides for a novel nutritional composition which promotes fast weight loss, burns calories, increases thermogenesis, supports energy metabolism and/or suppresses appetites in individuals on a low carbohydrate diet. In another embodiment, the present invention provides for a novel nutritional composition which promotes fast weight loss, burns calories, increases thermogenesis, supports energy metabolism and/or suppresses appetites in individuals on a reduced caffeine or caffeine-free dietary regimen. In still another embodiment, the present invention provides for a novel nutritional composition which promotes fast weight loss, burns calories, increases thermogenesis, supports energy metabolism and/or suppresses appetites in individuals, the nutritional composition providing natural ingredients.

The present invention provides for methods of inducing fast weight loss, burning calories, increasing thermogenesis, supports energy metabolism and/or suppressing appetite by consuming a novel nutritional composition which promotes fast weight loss, burns calories, increases thermogenesis, supports energy metabolism and/or suppresses appetites in individuals, the nutritional composition including, in one embodiment, garcinia cambogia extract, gymnema sylvestre leaf extract, and green tea leaf extract. In one embodiment, the present invention provides for a method inducing fast weight loss, burning calories, increasing thermogenesis, supports energy metabolism and/or suppressing appetite by consuming a novel nutritional composition which promotes fast weight loss, burns calories, increases thermogenesis, supports energy metabolism and/or suppresses appetites in individuals on a low carbohydrate diet, the nutritional composition including, in one embodiment, garcinia cambogia extract, gymnema sylvestre leaf extract, and green tea leaf extract. In another embodiment, the present invention provides for a method of inducing fast weight loss, burning calories, increasing thermogenesis, supports energy metabolism and/or suppressing appetite by consuming a novel nutritional composition which promotes fast weight loss, burns calories, increases thermogenesis, supports energy metabolism and/or suppresses appetites in individuals on a reduced caffeine or caffeine-free dietary regimen, the nutritional composition including, in one embodiment, garcinia cambogia extract, gymnema sylvestre leaf extract, and green tea leaf extract. In still another embodiment, the present invention provides for a method of inducing fast weight loss, burning calories, increasing thermogenesis, supports energy metabolism and/or suppressing appetite by consuming a novel nutritional composition which promotes fast weight loss, burns calories, increases thermogenesis, supports energy metabolism and/or suppresses appetites in individuals, the nutritional composition providing natural ingredients, the nutritional composition including, in one embodiment, garcinia cambogia extract, gymnema sylvestre leaf extract, and green tea leaf extract.

The present invention provides for a method of manufacturing a novel nutritional composition which promotes fast weight loss, burns calories, increases thermogenesis, supports energy metabolism and/or suppresses appetites in individuals, the nutritional composition including, in one embodiment, garcinia cambogia extract, gymnema sylvestre leaf extract, and green tea leaf extract. In one embodiment, the present invention provides for a method of manufacturing a novel nutritional composition which promotes fast weight loss, burns calories, increases thermogenesis, supports energy metabolism and/or suppresses appetites in individuals on a low carbohydrate diet, the nutritional composition including, in one embodiment, garcinia cambogia extract, gymnema sylvestre leaf extract, and green tea leaf extract. In another embodiment, the present invention provides for a method of manufacturing a novel nutritional composition which promotes fast weight loss, burns calories, increases thermogenesis, supports energy metabolism and/or suppresses appetites in individuals on a reduced caffeine or caffeine-free dietary regimen, the nutritional composition including, in one embodiment, garcinia cambogia extract, gymnema sylvestre leaf extract, and green tea leaf extract. In still another embodiment, the present invention provides for a method of manufacturing a novel nutritional composition which promotes fast weight loss, burns calories, increases thermogenesis, supports energy metabolism and/or suppresses appetites in individuals, the nutritional composition providing natural ingredients, the nutritional composition including, in one embodiment, garcinia cambogia extract, gymnema sylvestre leaf extract, and green tea leaf extract.

### DETAILED DESCRIPTION OF THE INVENTION

Definitions

As used herein, “an effective amount” refers to an amount effective for inducing weight loss when administered as a serving three times a day over a period of week(s). An effective amount of the nutritional composition is preferably from about 0.1 g to about 10 g of the nutritional composition per serving. More preferably, an effective amount of the composition comprises from about 1 g to about 5 g of the nutritional composition per serving. Most preferably, an effective amount of the composition comprises about 2.0 to about 2.6 g of the nutritional composition.

As used herein, “essentially pure” preferably refers to a compound that is greater than about 90% pure. More preferably, “essentially pure” refers to a compound that is greater than about 95% pure. Most preferably, “essentially pure” refers to a compound that is greater than about 98% pure.

As used herein, “enriched” refers to a partially purified extract or composition from which undesirable impurities have been removed. The undesirable impurities may be a single compound or multiple compounds. Preferably, “enriched” refers to a composition in which at least 25% of the undesirable impurities have been removed. More preferably, “enriched” refers to a composition in which at least 50% of the undesirable impurities have been removed. Most preferably, “enriched” refers to a composition in which at least 75% of the undesirable impurities have been removed.

The present invention, according to one embodiment, provides for a nutritional composition including garcinia cambogia extract, gymnema sylvestre leaf extract, and green tea leaf extract.

In one preferred embodiment, which is set forth in additional detail in Example 1, the present invention provides for a nutritional composition including garcinia cambogia (Super Citrimax 60% HCA); glucomannan (amorphophallus konjac); gymnema sylvestre leaf extract 25% gymnemic acid; alpha lipoic acid (“ALA”); chromate (10% elemental chromium); green tea—dry leaf extract (45% EGCG); anydrous caffeine; guarana extract (22% caffeine); cinnulin PF; griffonia simplicifolia 95% 5-hydroxytryptophan; banaba (standardized to 1% corosolic acid); and/or xanthan gum. This embodiment of the present invention may provide for a novel nutritional composition which promotes fast weight loss, burns calories, increases thermogenesis, supports energy metabolism and/or suppresses appetites in individuals.

In another preferred embodiment, which is set forth in additional detail in Example 3, the present invention provides for a nutritional composition including garcinia cambogia—dried fruit and rind extract; N-olyl-phosphatidyl ethanolamine (NOPE)/EGCG blend (from standard green tea extract, camellia sinensis); green tea dry leaf extract (camellia sinensis); white tea dry leaf extract (camellia sinensis); oolong tea dry leaf extract (camellia sinensis); anhydrous caffeine; gymnema sylvestre leaf extract; rhodiola rosea root extract; enriched soy phospholipid; withania somnifera root extract; chromium polynicotinate; and/or elemental chromium. This embodiment of the present invention may provide for a novel nutritional composition which promotes fast weight loss, burns calories, increases thermogenesis, supports energy metabolism and/or suppresses appetites in individuals.

In another preferred embodiment, which is set forth in additional detail in Example 5, the present invention provides for a nutritional composition including garcinia cambogia (dried fruit and rind extract); caffeine free green tea dry leaf extract (camellia sinensis); gymnema sylvestre leaf extract; N-olyl-phosphatidyl ethanolamine (NOPE)/EGCG blend (from std green tea extract, camellia sinensis); caffeine free white tea dry leaf extract (camellia sinensis); caffeine free oolong tea dry leaf extract (camellia sinensis); chromium polynicotinate; elemental chromium; rhodiola rosea root extract; enriched soy phospholipid; and/or withania somnifera root extract. This embodiment of the present invention may provide for a novel nutritional composition which promotes fast weight loss, burns calories, increases thermogenesis, supports energy metabolism and/or suppresses appetites in individuals on a reduced caffeine or caffeine-free dietary regimen.

In another preferred embodiment, which is set forth in additional detail in Example 7, the present invention provides for a nutritional composition including garcinia cambogia (dried fruit and rind extract); green tea dry leaf extract (camellia sinensis); anhydrous caffeine; gymnema sylvestre leaf extract; N-olyl-phosphatidyl ethanolamine (NOPE)/EGCG blend (from standard green tea extract, camellia sinensis); white tea dry leaf extract (camellia sinensis); oolong tea dry leaf extract (camellia sinensis); chromium polynicotinate; elemental chromium; rhodiola rosea root extract; enriched soy phospholipid; withania somnifera root extract; cinnamon bark extract (cinnamomum cassia); banaba leaf powder (lagerstroemia speciosa); and/or griffonia simplicifolia seed extract. This embodiment of the present invention may provide for a novel nutritional composition which promotes fast weight loss, burns calories, increases thermogenesis, supports energy metabolism and/or suppresses appetites in individuals on a low carbohydrate diet.

In another preferred embodiment, which is set forth in additional detail in Example 9, the present invention provides for a nutritional composition including garcinia cambogia (dried fruit and rind extract); guarana powder (from seed); green tea dry leaf extract (camellia sinensis); gymnema sylvestre leaf extract; N-olyl-phosphatidyl ethanolamine (NOPE)/EGCG blend (from standard green tea extract, camellia sinensis); white tea dry leaf extract (camellia sinensis); oolong tea dry leaf extract (camellia sinensis); rhodiola rosea root extract; enriched soy phospholipid; and/or withania somnifera root extract. This embodiment of the present invention may provide for a novel nutritional composition which promotes fast weight loss, burns calories, increases thermogenesis, supports energy metabolism and/or suppresses appetites in individuals the nutritional composition providing natural ingredients.

In another preferred embodiment, which is set forth in additional detail in Example 11, the present invention provides for a nutritional composition including garcinia cambogia , guarana seed (15% natural caffeine), green tea leaf, white willow bark, carnitine I-tartrate, chromium picolinate, magnesium stearate silicon dioxide fumed, and microcrystalline cellulose. This embodiment of the present invention may provide for a novel nutritional composition which promotes fast weight loss, burns calories, increases thermogenesis, supports energy metabolism and/or suppresses appetites in individuals the nutritional composition providing natural ingredients.

A discussion of some of these ingredients is set forth below:

Garcinia Cambogia

According to various embodiments of the present invention, the supplemental dietary composition includes garcinia cambogia (hydroxycitric acid and its calcium/potassium salt). Advantageously, these ingredients may be provided as, e.g., full strength Super CitriMax® from InterHealth N.I.

Super CitriMax® is an all-natural, safe and effective plant extract used as diet ingredients. Extracted from the South Asian fruit Garcinia cambogia, Super CitriMax® contain standardized levels of (−)hydroxycitric acid (HCA), which has been clinically shown to suppress appetite and inhibit fat production, without stimulating the central nervous system. More recently, researchers have shown that clinical strength doses of Super CitriMax® may be an even more powerful, all-natural diet aid for reducing or maintaining weight.

Garcinia cambogia is a natural source of hydroxycitrate. Although the present invention is not to be limited by any such theoretical explanations, it is believed that hydroxycitrate prevents the conversion of carbohydrates into fat by blocking the enzyme ATP citrate lyase and thus may induce weight loss and fat loss. It also improves satiety.

Preferably the dietary supplement of the present invention comprises sufficient garcinia cambogia to provide a daily dose of from about 1 to about 10,000 mg of hydroxycitrate. More preferably, the dietary supplement comprises sufficient garcinia cambogia to provide a daily dose of from about 5 to about 8000 mg of hydroxycitrate. Most preferably, the dietary supplement comprises sufficient garcinia cambogia to provide a daily dose of from about 10 to about 5000 mg of hydroxycitrate. According to one embodiment of the present invention, the dietary supplement includes about 1.555 g of garcinia cambogia salt per serving, consumed three times daily, for a daily intake of about 4.65 g.

Gymnema Sylvestre

Gymnema sylvestre is a woody climbing plant that grows in the tropical forests of central and southern India. The leaves are used in herbal medicine preparations and gymnema sylvestre leaf extract in the control of blood glucose in insulin-dependent diabetes mellitus. Gymnema sylvestre leaf extract preferably comprises about 25% gymnemic acid.

Preferably the dietary supplement of the present invention provides a daily dose of from about 1 to about 10,000 mg of gymnema sylvestre leaf extract. More preferably, the dietary supplement comprises sufficient garcinia cambogia to provides a daily dose of from about 5 to about 8,000 mg of gymnema sylvestre leaf extract. Most preferably, the dietary supplement comprises sufficient garcinia cambogia to provides a daily dose of from about 10 to about 5,000 mg of gymnema sylvestre leaf extract. According to one embodiment of the present invention, the dietary supplement includes about 0.133 g of gymnema sylvestre per serving, consumed three times daily, for a daily intake of about 0.399 g.

Green Tea Leaf Extract

In a preferred embodiment the nutritional composition comprises green tea leaf extract which preferably comprises caffeine, catechin-polyphenols, another methyl-xanthine and combinations thereof. In an alternative embodiment, a caffeine-free green tea leaf extract may be employed.

Preferably a serving of the nutritional composition comprises sufficient tea, green tea or as an enriched green tea extract to provide from about 25 to about 1000 mg of caffeine. More preferably, a serving of the nutritional composition comprises sufficient green tea or enriched green tea extract to provide from about 50 to about 250 mg of caffeine. Most preferably, a serving of the nutritional composition comprises sufficient green tea or enriched green tea extract to provide about 200 mg of caffeine.

Preferably a serving of the nutritional composition comprises sufficient tea, green tea or enriched green tea extract to provide from about 1 to about 1000 mg of a catechin-polyphenol. More preferably, a serving comprises sufficient green tea or enriched green tea extract to provide from about 75 to about 500 mg of catechin-polyphenol. Most preferably, a serving comprises sufficient green tea or enriched green tea extract to provide about 90 mg of catechin-polyphenol.

Caffeine may alternatively be supplied as essentially pure caffeine or as an ingredient naturally occurring in other ingredients. Catechin-polyphenol may also be supplied as an essentially pure catechin-polyphenol or as an enriched catechin-polyphenol. An essentially pure or enriched catechin-polyphenol may be selected from the group consisting of epigallocatechin-gallate, epicatechin-gallate, epicatechin, and epigallocatechin.

Optionally green tea is supplemented with essentially pure caffeine to supplement the thermogenic properties of green tea alone.

Optionally green tea is supplemented with an essentially pure catechin-polyphenol to supplement the thermogenic properties of green tea alone.

Dietary Fiber

In a preferred embodiment the nutritional composition comprises dietary fiber which optionally acts as a the satiety-promoting ingredient. Preferably, the dietary fiber comprises a mixture of glucomannan and a gum. The gum is preferably selected from the group consisting of xanthan gum, guar gum, carageenan, and locust beam gum. Most preferably the gum is xanthan gum. Alternatively, the dietary fiber may comprise a mixture of glucomannan and a blend of gums. Preferably the blend of gums comprises at least one compound selected from the group consisting of xanthan gum, guar gum, carageenan and locust bean gum.

Glucomannan, also known as konjak or konjak-mannan, is a highly viscous, water-soluble dietary fiber obtained by grinding the tuber root of the amorphophallus konjac (also known as proteinophallus rivieri) plant. Glucomannan is differentiated from other soluble fibers by the extraordinarily high viscosity of glucomannan solutions. Administration of 4-5 g of glucomannan with meals, blended into fluid or mixed with food, may slow carbohydrate absorption and may dampen the postprandial insulin response by up to 50%. Controlled clinical studies document that glucomannan may promote satiety and weight loss.

Xanthan gum is produced by industrial fermentation of a carbohydrate under aerobic conditions by culturing the bacterium xanthomonas campestris. It provides high viscosity solutions at low concentrations, exhibits little change in viscosity with variation in temperature, and is stable over a wide pH range. A common use of xanthan gum is in salad dressing, and it is also used in baked goods, confectionery products, syrups, toppings, dry beverage mixes, frozen foods, and dairy products. Xanthan gum is synergistic with locust bean gum and guar gum, and they are often used together for enhanced gelation or viscosity.

In the preferred embodiment, each serving comprises a mixture of glucomannan and xanthan gum at a weight ratio from about 1:1 to about 5:1. More preferably, each serving comprises glucomannan and xanthan gum at a weight ratio of about 4:1.

In one preferred embodiment, glucomannan is present in the nutritional composition in an amount from about 0.01 g to about 5 g per serving. More preferably, glucomannan is present in an amount from about 0.1 g to about 3 g per serving. Most preferably, glucomannan is present in an amount of about 0.50 g per serving.

In one preferred embodiment, xanthan gum is present in an amount from about 0.01 g to about 5 g per serving. More preferably the xanthan gum is present in an amount from about 0.1 g to about 3 g per serving. Most preferably, the xanthan gum is present in an amount of about 0.125 g per serving.

Guarana Extract

Caffeine may also be supplied from natural sources such as guarana. Guarana (Paullina Cupana) is nut-like seed of a climbing vine that grows in the Amazon basin in Brazil just south of the equator and is a natural source of caffeine. Guarana has been used for centuries by the indigenous people of this area as natural energy supplement.

In one preferred embodiment of the present invention, the nutritional composition comprises from about 1 mg to about 1000 mg of guarana extract. More preferably, guarana extract is present in an amount from about 1 mg to about 500 mg per serving. In one most preferred embodiment, guarana extract is present in an amount of about 680 mg per serving.

Cinnamomum Extract

Cinnamomum extract has been shown to lower blood sugar due to and help the body handling glucose and as such also effective in weight loss.

Preferaby the nutritional composition comprises from about 50 mg to about 1000 mg of cinnamomum extract. More preferably, the cinnamomum extract is present in an amount from about 75 mg to about 500 mg per serving. Most preferably, the cinnamomum extract is present in an amount of about 100 mg per serving.

Banaba Leaf (Lagestroemia Speciosa)

Banaba leaves have been shown to lower blood sugar due to and help the body handling glucose and as such also effective in weight loss.

Preferaby the nutritional composition comprises from about 50 mg to about 1000 mg of banaba leaf or banaba leaf extract. More preferably, the banaba leaf or banaba leaf extract is present in an amount from about 75 mg to about 500 mg per serving. Most preferably, the banaba leaf or banaba leaf extract is present in an amount of about 15 mg per serving.

Griffonia Simplicifolia

Griffonia simplicifolia seed are a natural source of 5- Hydroxytryptophane, which has been shown enhances the activity of serotonin, a hormone produced by the brain that is involved in mood, sleep and appetite.

Preferably the nutritional composition comprises from about 50 mg to about 1000 mg of griffonia simplicifolia seed extract. More preferably, the griffonia simplicifolia seed extract is present in an amount from about 75 mg to about 500 mg per serving. Most preferably, the griffonia simplicifolia is present in an amount of about 10 mg per serving.

Oolong Tea Dry Leaf Extract

Conventionally, oolong tea has been used to improve cognitive performance and mental alertness, to prevent cancer, tooth decay, and cardiovascular disease, and to treat atopic dermatitis, obesity, diabetes, atherosclerosis, and hyperlipidemia, and to improve resistance to disease.

Typically, the applicable parts of oolong tea are the leaf bud, leaf, and stem. Oolong tea is partially fermented, and flavanols include epigallocatechin gallate (EGCG), epigallocatechin (EGC), epicatechin gallate (ECG), epicatechin (EC), and gallocatechin gallate (GCG). Oolong tea contains about 23.2% catechins. Oolong tea may contain 50-60 mg caffeine per cup. The caffeine in oolong tea stimulates the central nervous system (CNS), heart, muscles, and possibly the pressor centers that control blood pressure. Possible mechanisms include adenosine receptor blockade and phosphodiesterase inhibition. The caffeine content is also thought to be responsible for oolong tea's effects on cognitive performance.

Oolong tea seems to have antioxidant properties. It seems to suppress the oxidation low-density lipoprotein (LDL) cholesterol. Oxidized LDL may contribute to atherosclerosis. Caffeine, a constituent of oolong tea, has been reported to cause increases and decreases in blood glucose. In people with type 2 diabetes, acute administration of caffeine impairs postprandial glucose metabolism, while acute abstention from caffeine reduces postprandial glucose levels by 21%.

Although the present invention is not to be limited by any theoretical explanation, it is believed that the caffeine in oolong tea may contribute to a weight loss effect, and other constituents may cause weight loss by inhibiting the activity of pancreatic lipase, which breaks down fat. Preliminary research suggests that oolong tea has an antiobesity effect in a high-fat diet. Preliminary clinical research suggests that oolong tea increases the metabolic rate and fat oxidation.

As used herein, a serving of the supplement comprises from about 0.00001 g to about 1 g of oolong tea dry leaf extract per serving. More preferably, a serving of the supplement comprises from about 0.0001 g to about 0.5 g of oolong tea dry leaf extract per serving. Most preferably, a serving of the supplement comprises about 0.005 g oolong tea dry leaf extract per serving.

White Tea Dry Leaf Extract

White tea is high in antioxidants and consists of young buds that are covered with white down. Researchers have compared the effects of white and green tea extracts at inactivating viruses and preventing the growth of bacteria, the study showing that the white tea extract was significantly more effective than the green at fighting germs. It is believed that white tea extract may have antiviral and antifungal effects and may be used in the future to develop treatments to help prevent disease.

As used herein, a serving of the supplement comprises from about 0.00001 g to about 1 g of white tea dry leaf extract per serving. More preferably, a serving of the supplement comprises from about 0.0001 g to about 0.5 g of white tea dry leaf extract per serving. Most preferably, a serving of the supplement comprises about 0.005 g white tea dry leaf extract per serving.

Rhodiola Rosea

Rhodiola rosea belongs to the plant family Crassulaceae and grows primarily in dry sandy ground at high altitudes in the arctic areas of Europe and Asia. Rhodiola rosea has been used in the traditional medicine of Russia, Scandinavia, and other countries. Medical and pharmacological texts describe its use as a stimulant for asthenia (fatigue), for somatic and infectious illnesses, in psychiatric and neurological conditions, and in healthy individuals to relieve fatigue and to increase attention span, memory, and work productivity. In addition, Rhodiola rosea has been recognized as an antifatigue agent, and as providing a stimulant action.

As used herein, a serving of the supplement comprises from about 0.00001 g to about 1 g of Rhodiola rosea per serving. More preferably, a serving of the supplement comprises from about 0.0001 g to about 0.5 g of Rhodiola rosea per serving. Most preferably, a serving of the supplement comprises about 0.001 g Rhodiola rosea per serving.

Withania Somnifera

Withania somnifera, or Ashwagandha, is native to drier parts of India. It is a perennial herb that contains the alkaloids withanine and somniferine, which are used to treat nervous disorders, intestinal infections and leprosy.

As used herein, a serving of the supplement comprises from about 0.00001 g to about 1 g of Withania somnifera per serving. More preferably, a serving of the supplement comprises from about 0.0001 g to about 0.5 g of Withania somnifera per serving. Most preferably, a serving of the supplement comprises about 0.001 g Withania somnifera per serving.

Chromium Polynicotinate

Chromium plays a role in the metabolism of glucose, and is necessary for energy production. Since this mineral assists in the production of insulin, it helps to stabilize blood sugar levels and can be beneficial both for people with hypoglycemia and diabetes. It is also critical to the synthesis of cholesterol, fats, and proteins.

Chromium polynicotinate is more effective than any other type of chromium, as it binds the elemental chromium to niacin (vitamin B-3). This provides a biologically active form of chromium, which is more absorbable in the body.

Although the present invention is not to be limited by any theoretical explanation, it is believed that chromium is mainly used to stabilize blood sugar. The major role for chromium in weight loss is to correct insulin resistance caused by chromium deficiency. Studies have shown that chromium is at the heart of a biologically active complex called Glucose Tolerance Factor or “GTF”, which is responsible for potentiating (increasing) the action of insulin in the body. Insulin is a vital hormone responsible for transporting glucose inside cells and maintaining proper blood sugar and blood lipid (cholesterol) levels in the body. Chromium helps insulin metabolize fat, turn protein into muscle, and convert sugar into energy.

As used herein, a serving of the supplement comprises from about 0.00001 g to about 1 g of chromium polynicotinate per serving. More preferably, a serving of the supplement comprises from about 0.0001 g to about 0.5 g of chromium polynicotinate per serving. Most preferably, a serving of the supplement comprises about 0.00133 g chromium polynicotinate per serving.

The dosage form of the nutritional supplement is preferably provided as a caplet. However, the dosage form of the dietary supplement may alternatively be provided as a capsule, a liquid beverage, a powder beverage mix, as a ready-to-eat bar product, or in any other form.

The nutritional compositions of the present invention may be provided in accordance with customary processing techniques for herbal, dietary supplements wherein the active ingredients are suitably processed and compressed into a caplet with suitable excipients. For instance, the nutritional compositions of the present invention may be processed and compressed into caplets with cellulose, croscarmellose sodium, stearic acid, magnesium stearate, silica and other with suitable excipients. Those of skill in the art will appreciate that the supplemental composition may contain a variety of excipients.

The present invention provides for methods of inducing fast weight loss, burning calories, increasing thermogenesis, supports energy metabolism and/or suppressing appetite by consuming an effective amount of a novel nutritional composition, which promotes fast weight loss, burns calories, increases thermogenesis, supports energy metabolism and/or suppresses appetites in individuals. In one such embodiment, the present invention provides for methods of inducing fast weight loss, burning calories, increasing thermogenesis, supports energy metabolism and/or suppressing appetite by consuming an effective amount of a novel nutritional composition that includes garcinia cambogia (Super Citrimax 60% HCA); glucomannan (amorphophallus konjac); gymnema sylvestre leaf extract 25% gymnemic acid; alpha lipoic acid (“ALA”); chromate (10% elemental chromium); green tea—dry leaf extract (45% EGCG); anydrous caffeine; guarana extract (22% caffeine); cinnulin PF; griffonia simplicifolia 95% 5-hydroxytryptophan; banaba (standardized to 1% corosolic acid); and/or xanthan gum. In another such embodiment, the present invention provides for methods of inducing fast weight loss, burning calories, increasing thermogenesis, supports energy metabolism and/or suppressing appetite by consuming an effective amount of a novel nutritional composition that includes garcinia cambogia—dried fruit and rind extract; N-olyl-phosphatidyl ethanolamine (NOPE)/EGCG blend (from standard green tea extract, camellia sinensis); green tea dry leaf extract (camellia sinensis); white tea dry leaf extract (camellia sinensis); oolong tea dry leaf extract (camellia sinensis); anhydrous caffeine; gymnema sylvestre leaf extract; rhodiola rosea root extract; enriched soy phospholipid; withania somnifera root extract; chromium polynicotinate; and/or elemental chromium.

In one embodiment, the present invention provides for a method inducing fast weight loss, burning calories, increasing thermogenesis, supports energy metabolism and/or suppressing appetite by consuming an effective amount of a novel nutritional composition which promotes fast weight loss, burns calories, increases thermogenesis, supports energy metabolism and/or suppresses appetites in individuals on a low carbohydrate diet. In one such embodiment, the present invention provides for methods of inducing fast weight loss, burning calories, increasing thermogenesis, supports energy metabolism and/or suppressing appetite by consuming an effective amount of a novel nutritional composition that includes garcinia cambogia (dried fruit and rind extract); green tea dry leaf extract (camellia sinensis); anhydrous caffeine; gymnema sylvestre leaf extract; N-olyl-phosphatidyl ethanolamine (NOPE)/EGCG blend (from standard green tea extract, camellia sinensis); white tea dry leaf extract (camellia sinensis); oolong tea dry leaf extract (camellia sinensis); chromium polynicotinate; elemental chromium; rhodiola rosea root extract; enriched soy phospholipid; withania somnifera root extract; cinnamon bark extract (cinnamomum cassia); banaba leaf powder (lagerstroemia speciosa); and/or griffonia simplicifolia seed extract.

In another embodiment, the present invention provides for a method of inducing fast weight loss, burning calories, increasing thermogenesis, supports energy metabolism and/or suppressing appetite by consuming an effective amount of a novel nutritional composition which promotes fast weight loss, burns calories, increases thermogenesis, supports energy metabolism and/or suppresses appetites in individuals on a reduced caffeine or caffeine-free dietary regimen. In one such embodiment, the present invention provides for methods of inducing fast weight loss, burning calories, increasing thermogenesis, supports energy metabolism and/or suppressing appetite by consuming an effective amount of a novel nutritional composition that includes garcinia cambogia (dried fruit and rind extract); caffeine free green tea dry leaf extract (camellia sinensis); gymnema sylvestre leaf extract; N-olyl-phosphatidyl ethanolamine (NOPE)/EGCG blend (from std green tea extract, camellia sinensis); caffeine free white tea dry leaf extract (camellia sinensis); caffeine free oolong tea dry leaf extract (camellia sinensis); chromium polynicotinate; elemental chromium; rhodiola rosea root extract; enriched soy phospholipid; and/or withania somnifera root extract.

In still another embodiment, the present invention provides for a method of inducing fast weight loss, burning calories, increasing thermogenesis, supports energy metabolism and/or suppressing appetite by consuming an effective amount of a novel nutritional composition which promotes fast weight loss, burns calories, increases thermogenesis, supports energy metabolism and/or suppresses appetites in individuals, the nutritional composition providing natural ingredients. In one such embodiment, the present invention provides for methods of inducing fast weight loss, burning calories, increasing thermogenesis, supports energy metabolism and/or suppressing appetite by consuming an effective amount of a novel nutritional composition that includes garcinia cambogia (dried fruit and rind extract); guarana powder (from seed); green tea dry leaf extract (camellia sinensis); gymnema sylvestre leaf extract; N-olyl-phosphatidyl ethanolamine (NOPE)/EGCG blend (from standard green tea extract, camellia sinensis); white tea dry leaf extract (camellia sinensis); oolong tea dry leaf extract (camellia sinensis); rhodiola rosea root extract; enriched soy phospholipid; and/or withania somnifera root extract.

The nutritional composition is optionally administered to any animal for inducing fast weight loss, burning calories, increasing thermogenesis, supports energy metabolism and/or suppressing appetite. The animal is preferably a human. The human may be an athlete or may be a non-athlete.

In a preferred method of inducing fast weight loss, burning calories, increasing thermogenesis, supports energy metabolism and/or suppressing appetite, the nutritional composition is administered between 1 and 4 times daily. The nutritional composition is optionally administered at meals. Preferably, the nutritional composition is administered three times a day. More preferably, the nutritional composition is administered three times a day for a period of at least about a week. While the present invention is intended to cover the use of the nutritional composition for any period of time, it is even more preferable that the nutritional composition is administered three times a day for a period of about at least 8 weeks, although periods of time longer than 8 weeks are also contemplated. In one embodiment, a serving is two caplets.

In a preferred method of inducing fast weight loss, burning calories, increasing thermogenesis, supports energy metabolism and/or suppressing appetite, the nutritional composition is administered to an individual on a reduced calorie diet, for instance, a diet consisting of no more than 2000 calories. In another preferred method of inducing fast weight loss, burning calories, increasing thermogenesis, supports energy metabolism and/or suppressing appetite, the nutritional composition is administered to an individual engaging in at least moderate exercise regimen. It is believed that the consumption of nutritional compositions in accordance with various embodiments of the present invention as set forth herein may result in the loss of body weight at two to three times the rate provided by diet and exercise alone.

The present invention also provides for a method of making a nutritional composition. In accordance with one such embodiment, the method of making a nutritional composition includes the steps of: premixing a chromium chelate with microcrystalline cellulose; adding the following ingredients to the premix: garcinia cambogia extract, gymnema sylvestre leaf extract, glucomannan, chromium chelate, alpha lipoic acid, green tea extract (leaf, caffeine, guarana extract (seed), cinnamomum verum extract (bark), xanthan gum, banaba (lagestroemia speciosa) (leaf), and griffonia simplicifolia (seed); and in an additional step adding magnesium stearate and silica which had been pre-sifted; blending and mixing for 30 minutes. Finally, the method includes the step of checking for uniformity/homogeneity and then aliquoting into a serving for compression into a caplet.

In accordance with another such embodiment, the method of making a nutritional composition includes the steps of: premixing a chromium chelate with microcrystalline cellulose; adding the following ingredients to the premix: garcinia cambogia—dried fruit and rind extract; N-olyl-phosphatidyl ethanolamine (NOPE)/EGCG blend (from standard green tea extract, camellia sinensis); green tea dry leaf extract (camellia sinensis); white tea dry leaf extract (camellia sinensis); oolong tea dry leaf extract (camellia sinensis); anhydrous caffeine; gymnema sylvestre leaf extract; rhodiola rosea root extract; enriched soy phospholipid; withania somnifera root extract; chromium polynicotinate; and/or elemental chromium; and in an additional step adding the following ingredients magnesium stearate and silica which had been pre-sifted; blending and mixing for 30 minutes. Finally, the method includes the step of checking for uniformity/homogeneity and then aliquoting into a serving for compression into a caplet.

In accordance with another such embodiment, the method of making a nutritional composition includes the steps of: premixing a chromium chelate with microcrystalline cellulose; adding the following ingredients to the premix: garcinia cambogia (dried fruit and rind extract); caffeine free green tea dry leaf extract (camellia sinensis); gymnema sylvestre leaf extract; N-olyl-phosphatidyl ethanolamine (NOPE)/EGCG blend (from std green tea extract, camellia sinensis); caffeine free white tea dry leaf extract (camellia sinensis); caffeine free oolong tea dry leaf extract (camellia sinensis); chromium polynicotinate; elemental chromium; rhodiola rosea root extract; enriched soy phospholipid; and/or withania somnifera root extract; and in an additional step adding the following ingredients magnesium stearate and silica which had been pre-sifted; blending and mixing for 30 minutes. Finally, the method includes the step of checking for uniformity/homogeneity and then aliquoting into a serving for compression into a caplet.

In accordance with another such embodiment, the method of making a nutritional composition includes the steps of: premixing a chromium chelate with microcrystalline cellulose; adding the following ingredients to the premix: garcinia cambogia (dried fruit and rind extract); green tea dry leaf extract (camellia sinensis); anhydrous caffeine; gymnema sylvestre leaf extract; N-olyl-phosphatidyl ethanolamine (NOPE)/EGCG blend (from standard green tea extract, camellia sinensis); white tea dry leaf extract (camellia sinensis); oolong tea dry leaf extract (camellia sinensis); chromium polynicotinate; elemental chromium; rhodiola rosea root extract; enriched soy phospholipid; withania somnifera root extract; cinnamon bark extract (cinnamomum cassia); banaba leaf powder (lagerstroemia speciosa); and/or griffonia simplicifolia seed extract; and in an additional step adding the following ingredients magnesium stearate and silica which had been pre-sifted; blending and mixing for 30 minutes. Finally, the method includes the step of checking for uniformity/homogeneity and then aliquoting into a serving for compression into a caplet.

In accordance with another such embodiment, the method of making a nutritional composition includes the steps of: premixing a chromium chelate with microcrystalline cellulose; adding the following ingredients to the premix: garcinia cambogia (dried fruit and rind extract); guarana powder (from seed); green tea dry leaf extract (camellia sinensis); gymnema sylvestre leaf extract; N-olyl-phosphatidyl ethanolamine (NOPE)/EGCG blend (from standard green tea extract, camellia sinensis); white tea dry leaf extract (camellia sinensis); oolong tea dry leaf extract (camellia sinensis); rhodiola rosea root extract; enriched soy phospholipid; and/or withania somnifera root extract; and in an additional step adding the following ingredients magnesium stearate and silica which had been pre-sifted; blending and mixing for 30 minutes. Finally, the method includes the step of checking for uniformity/homogeneity and then aliquoting into a serving.

Although the following examples illustrate the practice of the present invention in some of its embodiments, the examples should not be construed as limiting the scope of the invention. Other embodiments will be apparent to one skilled in the art from consideration of the specification and examples.

### EXAMPLE 1

A serving of the nutritional supplement comprises the following ingredients in caplet form.

| Ingredients | 2.842 g/serving | Formula % |
|---|---|---|
| Garcinia cambogia | 1.555 | 54.707% |
| (Super Citrimax 60% HCA) | | |
| Glucomannan | 0.50000 | 17.5907% |
| (Amorphophallus konjac) | | |
| Gymnema sylvestre leaf | 0.13330 | 4.6897% |
| extract 25% gymnemic acid | | |
| ALA | 0.01000 | 0.3518% |
| Chromate | 0.00133 | 0.0468% |
| (10% elemental chromium) | | |
| Green Tea - dry leaf extract | 0.20000 | 7.0363% |
| (45% EGCG) | | |
| Anydrous Caffeine | 0.19178 | 6.7471% |
| Guarana Extract (22% Caffeine) | 0.00100 | 0.0352% |
| Cinnulin PF | 0.10000 | 3.5181% |
| Griffonia simplicifolia 95% | 0.01000 | 0.3518% |
| 5-hydroxytryptophan | | |
| Banaba (standardized to 1% | 0.01500 | 0.5277% |
| corosolic acid | | |
| Xanthan gum | 0.12500 | 4.3977% |
| Total | 2.84241 | 100.000% |

### EXAMPLE 2

One serving, which consists of four caplets, of the supplement of Example 1 is consumed by a human on a low carbohydrate diet, 3 times a day for a period of a week.

### EXAMPLE 3

A serving of the nutritional supplement comprises the following ingredients in caplet form.

| | | Dietary | | |
|---|---|---|---|---|
| | | Ingredient | Actives/ | |
| Dietary Ingredient | Actives | per serv- | serving | |
| Name | % | ing (g) | (g) | Formula % |
| Garcinia cambogia - | | 1.55500 | | 73.6154% |
| Dried Fruit and Rind | | | | |
| Extract | | | | |
| Std to HCA | 60 | | | |
| N-olyl-phosphatidyl | | 0.01000 | | 0.4734% |
| ethanolamine (NOPE)/ | | | | |
| EGCG blend (from std | | | | |
| green tea extract, | | | | |
| Camellia sinensis) | | | | |
| Std to NOPE | 23 | | 0.002300 | |
| Std to Polyphenols | 20 | | 0.002000 | |
| Std to EGCG | 14 | | 0.001400 | |
| Green tea dry leaf | | 0.20000 | | 9.4682% |
| extract (Camellia | | | | |
| sinensis) | | | | |
| Std to EGCG | 45 | | 0.090000 | |
| Std to Catechins | 75 | | 0.150000 | |
| Std to Polyphenols | 90 | | 0.180000 | |
| White tea dry leaf | | 0.00500 | | 0.2367% |
| extract (Camellia | | | | |
| sinensis) | | | | |
| Std to EGCG | 15 | | 0.000750 | |
| Std to Catechins | 35 | | 0.001750 | |
| Std to Polyphenols | 50 | | 0.002500 | |
| Oolong tea dry leaf | | 0.00500 | | 0.2367% |
| extract (Camellia | | | | |
| sinensis) | | | | |
| Std to EGCG | 15 | | 0.000750 | |
| Std to Catechins | 25 | | 0.001250 | |
| Std to Polyphenols | 50 | | 0.002500 | |
| Anhydrous caffeine | | 0.20000 | | 9.4682% |
| Gymnema sylvestre | | 0.13300 | | 6.2964% |
| leaf extract | | | | |
| Std to Gymnemic | 25 | | 0.033250 | |
| Acid | | | | |
| Rhodiola Rosea | | 0.00100 | 0.000000 | 0.0473% |
| root extract | | | | |
| std to Total | 5 | | 0.000050 | |
| Rosavins | | | | |
| Enriched Soy | | 0.00100 | | 0.0473% |
| Phospholipid | | | | |
| Std to Phosphatidyl | 50 | | 0.000500 | |
| Serine | | | | |
| Std to Phosphatidyl | 4 | | 0.000040 | |
| Choline | | | | |
| Std to Phosphatidyl | 2 | | 0.000020 | |
| ethanolamine | | | | |
| Withania Somnifera | | 0.00100 | | 0.0473% |
| root extract | | | | |
| Std to Withanolides | 1.5 | | 0.000015 | |
| Chromium | | 0.00133 | | 0.0630% |
| Polynicotinate | | | | |
| Elemental Chromium | 10 | | 0.000133 | |
| | | 2.1123 | | 100.0000% |

### EXAMPLE 4

One serving of the supplement of Example 3 is consumed by a human, 3 times a day, each serving including 2 caplets.

### EXAMPLE 5

A serving of the nutritional supplement comprises the following ingredients in caplet form.

| | | Dietary | | |
|---|---|---|---|---|
| | | Ingredient | Actives/ | |
| Dietary Ingredient | Actives | per serving | serving | Formula |
| Name | % | (g) | (g) | % |
| Garcinia Cambogia | | 1.555 | | 81.3144% |
| (Dried fruit and | | | | |
| rind extract) | | | | |
| Std to HCA | 60 | | 0.93300 | |
| Caffeine Free | | 0.200 | | 10.4584% |
| Green Tea dry leaf | | | | |
| extract (Camellia | | | | |
| sinensis) | | | | |
| Std to EGCG | 45 | | 0.09000 | |
| Std to Catechins | 75 | | 0.15000 | |
| Std to Polyphenols | 90 | | 0.18000 | |
| Gymnema sylvestre | | 0.133 | | 6.9549% |
| leaf extract | | | | |
| Std to Gymnemic | 25 | | 0.03325 | |
| Acid | | | | |
| N-olyl-phosphatidyl | | 0.01 | | 0.5229% |
| ethanolamine (NOPE)/ | | | | |
| EGCG blend (from std | | | | |
| green tea extract, | | | | |
| Camellia sinensis) | | | | |
| Std to NOPE | 23 | | 0.00230 | |
| Std to Polyphenols | 20 | | 0.00200 | |
| Std to EGCG | 14 | | 0.00140 | |
| Caffeine Free White | | 0.005 | | 0.2615% |
| Tea dry leaf extract | | | | |
| (Camellia sinensis) | | | | |
| Std to EGCG | 15 | | 0.00075 | |
| Std to Catechins | 35 | | 0.00175 | |
| Std to Polyphenols | 50 | | 0.00250 | |
| Caffeine Free Oolong | | 0.005 | | 0.2615% |
| Tea dry leaf extract | | | | |
| (Camellia sinensis) | | | | |
| Std to EGCG | 15 | | 0.00075 | |
| Std to Catechins | 25 | | 0.00125 | |
| Std to Polyphenols | 50 | | 0.00250 | |
| Chromium | | 0.00133 | | 0.0695% |
| Polynicotinate | | | | |
| Elemental Chromium | 10 | | 0.00013 | |
| Rhodiola Rosea root | | 0.001 | | 0.0523% |
| extract | | | | |
| Std to Total | 5 | | 0.00005 | |
| Rosavins | | | | |
| Enriched Soy | | 0.001 | | 0.0523% |
| Phospholipid | | | | |
| Std to Phosphatidyl | 50 | | 0.00050 | |
| Serine | | | | |
| Std to Phosphatidyl | 4 | | 0.00004 | |
| Choline | | | | |
| Std to Phosphatidyl | 2 | | 0.00002 | |
| ethanolamine | | | | |
| Withania Somnifera | | 0.001 | | 0.0523% |
| Root extract | | | | |
| Std to Withanolides | 1.5 | | 0.00002 | |
| | | 1.9123 | | 100.0000% |

### EXAMPLE 6

One serving of the supplement of Example 5 is consumed by a human, 3 times a day, each serving including 2 caplets.

### EXAMPLE 7

A serving of the nutritional supplement comprises the following ingredients in caplet form.

| | | Dietary | | |
|---|---|---|---|---|
| | | Ingredient | Actives/ | |
| Dietary Ingredient | Actives | per serv- | serving | Formula |
| Name | % | ing (g) | (g) | % |
| Garcinia Cambogia | | 1.555 | | 75.9526% |
| (Dried fruit and | | | | |
| rind extract) | | | | |
| Std to HCA | 60 | | 0.93300 | |
| Green Tea dry leaf | | 0.01 | | 0.4884% |
| extract (Camellia | | | | |
| sinensis) | | | | |
| Std to EGCG | 45 | | 0.00450 | |
| Std to Catechins | 75 | | 0.00750 | |
| Std to Polyphenols | 90 | | 0.00900 | |
| Anhydrous caffeine | | 0.2 | | 9.7688% |
| Gymnema sylvestre | | 0.133 | | 6.4963% |
| leaf extract | | | | |
| Std to Gymnemic | 25 | | 0.03325 | |
| Acid | | | | |
| N-olyl-phospha- | | 0.01 | | 0.4884% |
| tidyl ethanolamine | | | | |
| (NOPE)/EGCG blend | | | | |
| (from std green tea | | | | |
| extract, camellia | | | | |
| sinensis) | | | | |
| Std to NOPE | 23 | | 0.00230 | |
| Std to Polyphenols | 20 | | 0.00200 | |
| Std to EGCG | 14 | | 0.00140 | |
| White Tea dry leaf | | 0.005 | | 0.2442% |
| extract (Camellia | | | | |
| sinensis) | | | | |
| Std to EGCG | 15 | | 0.00075 | |
| Std to Catechins | 35 | | 0.00175 | |
| Std to Polyphenols | 50 | | 0.00250 | |
| Oolong Tea dry leaf | | 0.005 | | 0.2442% |
| extract (Camellia | | | | |
| sinensis) | | | | |
| Std to EGCG | 15 | | 0.00075 | |
| Std to Catechins | 25 | | 0.00125 | |
| Std to Polyphenols | 50 | | 0.00250 | |
| Chromium | | 0.00133 | | 0.0650% |
| Polynicotinate | | | | |
| Elemental Chromium | 10 | | 0.000133 | |
| Rhodiola Rosea | | 0.001 | | 0.0488% |
| root extract | | | | |
| Std to Total | 5 | | 0.000050 | |
| Rosavins | | | | |
| Enriched Soy | | 0.001 | | 0.0488% |
| Phospholipid | | | | |
| Std to Phosphatidyl | 50 | | 0.000500 | |
| Serine | | | | |
| Std to Phosphatidyl | 4 | | 0.000040 | |
| Choline | | | | |
| Std to Phosphatidyl | 2 | | 0.000020 | |
| ethanolamine | | | | |
| Withania Somnifera | | 0.001 | | 0.0488% |
| root extract | | | | |
| Std to Withanolides | 1.5 | | 0.000015 | |
| Cinnamon bark | | 0.1 | | 4.8844% |
| extract (Cinnamomum | | | | |
| cassia) | | | | |
| Std to Polyphenols | 0.4 | | 0.00040 | |
| Std to MHCP | 2 | | 0.00200 | |
| Banaba Leaf Powder | | 0.015 | | 0.7327% |
| (Lagerstroemia | | | | |
| speciosa) | | | | |
| Supplying Corosolic | 1 | | 0.00015 | |
| Acid | | | | |
| Griffonia | | 0.01 | | 0.4884% |
| simplicifolia seed | | | | |
| extract | | | | |
| Std to 5-HTP | 95 | 2.0473 | 0.00950 | 100.0000% |
| | | 2.0473 | | 100.0000% |

### EXAMPLE 8

One serving of the supplement of Example 7 is consumed by a human, 3 times a day, each serving including 2 caplets.

### EXAMPLE 9

A serving of the nutritional supplement comprises the following ingredients in caplet form.

| | | Dietary | | |
|---|---|---|---|---|
| | | Ingredient | Actives/ | |
| Dietary Ingredient | Actives | per serv- | serving | Formula |
| Name | % | ing (g) | (g) | % |
| Garcinia Cambogia | | 1.55500 | | 59.9657% |
| (Dried fruit and | | | | |
| rind extract) | | | | |
| Std to HCA | 60 | | 0.93300 | |
| Guarana Powder | | 0.68182 | | 26.2931% |
| (from seed) | | | | |
| Std to Caffeine | 1 | | 0.00682 | |
| Std to Natural | 21 | | 0.14318 | |
| Caffeine | | | | |
| Green Tea dry leaf | | 0.20000 | | 7.7126% |
| extract (Camellia | | | | |
| sinensis) | | | | |
| Std to EGCG | 45 | | 0.09000 | |
| Std to Catechins | 75 | | 0.15000 | |
| Std to Polyphenols | 90 | | 0.18000 | |
| Gymnema sylvestre | | 0.13333 | | 5.1416% |
| leaf extract | | | | |
| Std to Gymnemic | 25 | | 0.03333 | |
| Acid | | | | |
| N-olyl-phospha- | | 0.01000 | | 0.3856% |
| tidyl ethanolamine | | | | |
| (NOPE)/EGCG blend | | | | |
| (from std green | | | | |
| tea extract, | | | | |
| Camellia sinensis) | | | | |
| Std to NOPE | 23 | | 0.00230 | |
| Std to Polyphenols | 20 | | 0.00200 | |
| Std to EGCG | 14 | | 0.00140 | |
| White Tea dry leaf | | 0.00500 | | 0.1928% |
| extract (Camellia | | | | |
| sinensis) | | | | |
| Std to EGCG | 15 | | 0.00075 | |
| Std to Catechins | 35 | | 0.00175 | |
| Std to Polyphenols | 50 | | 0.00250 | |
| Oolong Tea dry | | 0.00500 | | 0.1928% |
| leaf extract | | | | |
| (Camellia sinensis) | | | | |
| Std to EGCG | 15 | | 0.00075 | |
| Std to Catechins | 25 | | 0.00125 | |
| Std to Polyphenols | 50 | | 0.00250 | |
| Rhodiola Rosea | | 0.00100 | | 0.0386% |
| root extract | | | | |
| Std to Total | 5 | | 0.00005 | |
| Rosavins | | | | |
| Enriched Soy | | 0.00100 | | 0.0386% |
| Phospholipid | | | | |
| Std to Phosphatidyl | 50 | | 0.00050 | |
| Serine | | | | |
| Std to Phosphatidyl | 4 | | 0.00004 | |
| Choline | | | | |
| Std to Phosphatidyl | 2 | | 0.00002 | |
| ethanolamine | | | | |
| Withania Somnifera | | 0.00100 | | 0.0386% |
| root extract | | | | |
| Std to Withanolides | 1.5 | | 0.000015 | |
| | | 2.5931 | | 100.0000% |

### EXAMPLE 10

One serving of the supplement of Example 9 is consumed by a human, 3 times a day, each serving including 2 caplets.

### EXAMPLE 11

A serving of the nutritional supplement comprises the following ingredients in caplet form.

| | | Dietary | |
|---|---|---|---|
| | | Ingredient | Actives/ |
| | Actives | per serving | serving |
| Dietary Ingredient Name | % | (mg) | (mg) |
| Garcinia Cambogia (Fruit | 55 | 1818 | 1000 |
| SE 55% HCA Gran) | | | |
| Guarana Seed SE 15% | 15 | 1266 | 190 |
| Natural Caffeine | | | |
| Green Tea leaf SE 90PP | 5 | 200 | 10 |
| 75CAT 45EGCG 5CAFF | | | |
| White Willow Bark SE | 15 | 100 | 15 |
| 15% Salicin | | | |
| Carnitine I-Tartrate | 100 | 100 | 100 |
| I 68% | | | |
| Chromium Picolinate | 12 | 1.667 | 200 |
| 12% Cr | | | |
| Magnesium Stearate NF | 100 | 7.5 | 7.5 |
| Veg Source | | | |
| Silicon Dioxide Fumed | 100 | 2.225 | 2.25 |
| Microcrystalline Cellulose | 100 | 5.0 | 5 |
| PH102 Gran USP FCC | | | |
| | | 3500 | |

1-18. (canceled)

19. A method for promoting fast weight loss, burning calories, increasing thermogenesis, supports energy metabolism and/or suppressing appetite in individuals, the method comprising the step of:
consuming a nutritional composition comprising garcinia cambogia extract, gymnema sylvestre leaf extract, and green tea leaf extract.

20. The method of claim 19, wherein the nutritional composition is consumed by an individual on a low carbohydrate diet.

21. The method of claim 20, wherein the nutritional composition consumed further comprises at least one of anhydrous caffeine; N-olyl-phosphatidyl ethanolamine (NOPE)/EGCG blend; white tea dry leaf extract; oolong tea dry leaf extract; chromium polynicotinate; elemental chromium; rhodiola rosea root extract; enriched soy phospholipid; withania somnifera root extract; cinnamon bark extract; banaba leaf powder; and griffonia simplicifolia seed extract.

22. The method of claim 19, wherein the nutritional composition is consumed by an individual on a reduced caffeine or caffeine-free diet.

23. The method of claim 22, wherein the nutritional composition consumed further comprises at least one of caffeine free green tea dry leaf extract; N-olyl-phosphatidyl ethanolamine (NOPE)/EGCG blend; caffeine free white tea dry leaf extract; caffeine free oolong tea dry leaf extract; chromium polynicotinate; elemental chromium; rhodiola rosea root extract; enriched soy phospholipid; and withania somnifera root extract.

24. The method of claim 19, wherein the nutritional composition is consumed by an individual on a diet limited to natural ingredients.

25. The method of claim 24, wherein the nutritional composition consumed further comprises at least one of guarana powder; N-olyl-phosphatidyl ethanolamine (NOPE)/EGCG blend; white tea dry leaf extract; oolong tea dry leaf extract; rhodiola rosea root extract; enriched soy phospholipid; and withania somnifera root extract.
